(19) United States
(12) Patent Application Publication
(10) Pub. No.: US 2008/0281372 A1
(43) Pub. Date: Nov. 13, 2008

(54) NEURAL STIMULATION SYSTEM ANALYZER

(75) Inventors: Imad Libbus, St. Paul, MN (US); Avram Scheiner, Vadnais Heights, MN (US)

Correspondence Address: SCHWEGMAN, LUNDBERG & WOESSNER, P.A., P.O. BOX 2938, MINNEAPOLIS, MN, 55402, US

(73) Assignee: CARDIAC PACEMAKERS, INC., ST. PAUL, MN (US)

(21) Appl. No.: 11/746,263

(22) Filed: May 9, 2007

(51) Int. Cl. A61N 1/362

(52) U.S. Cl. 607/27

(57) ABSTRACT

Various embodiments relate to a device to analyze an implantable neural stimulation system that includes an implantable neural stimulation lead for an implantable neural stimulator to be implanted into a patient. Various device embodiments comprise an external housing, a pacing circuit in the housing, and a sensing circuit in the housing. The pacing circuit is adapted to deliver a test neural stimulation signal. At least one test lead cable is adapted to electrically connect the pacing circuit and the implantable neural stimulation lead to enable the test neural stimulation signal to be delivered to a neural target through the test lead cable and the implantable neural stimulation lead. At least one physiological sensor is adapted to sense a physiological response to stimulation of the neural target. At least one sensor cable is adapted to electrically connect the sensing circuit and the at least one physiological sensor.

### TECHNICAL FIELD

This application relates generally to medical devices and, more particularly, to systems, devices and methods for analyzing neural stimulation systems.

### BACKGROUND

Neural stimulation has been proposed to treat a number conditions. For example, vagal stimulation has been proposed to treat cardiovascular conditions such as heart failure, post-MI remodeling, hypertension, tachyarrhythmias, and atherosclerosis. Vagal stimulation has also been proposed to treat non-cardiovascular conditions such as epilepsy, depression, pain, obesity and diabetes. Implantable devices can be used to deliver neural stimulation.

### SUMMARY

Various embodiments relate to a device to analyze an implantable neural stimulation system that includes an implantable neural stimulation lead for an implantable neural stimulator to be implanted into a patient. Various device embodiments comprise an external housing, a pacing circuit in the housing, and a sensing circuit in the housing. The pacing circuit is adapted to deliver a test neural stimulation signal. At least one test lead cable is adapted to electrically connect the pacing circuit and the implantable neural stimulation lead to enable the test neural stimulation signal to be delivered to a neural target through the test lead cable and the implantable neural stimulation lead. At least one physiological sensor is adapted to sense a physiological response to stimulation of the neural target. At least one sensor cable is adapted to electrically connect the sensing circuit and the at least one physiological sensor.

Various device embodiments comprise an external housing, a pacing circuit in the housing, where the pacing circuit is adapted to deliver a test neural stimulation signal. At least one test lead cable is adapted to electrically connect the pacing circuit and the implantable neural stimulation lead to enable the test neural stimulation signal to be delivered to a vagus nerve through the test lead cable and the implantable neural stimulation lead. The at least one test lead cable includes at least one clip to connect to at least one terminal of the neural stimulation lead. The device includes a sensing circuit in the housing, a plurality of ECG electrodes adapted for use in detecting an electrocardiogram, and at least one sensor cable adapted to electrically connect the sensing circuit and the plurality of ECG electrodes. A controller is adapted to communicate with the pacing circuit and the sensing circuit to process the electrocardiogram for use in identifying a response to stimulation of the vagus nerve.

Various system embodiments for analyzing an implantable neural stimulation system comprise means for connecting a test lead cable for an external analyzer to at least one terminal of an implantable neural stimulation lead, means for delivering test neural stimulation using an external neural stimulation through the test lead cable and the neural stimulation to a neural target, and means for monitoring a physiologic response to the test neural stimulation.

An embodiment relates to a method for analyzing an implantable neural stimulation system. A neural stimulation lead is implanted. The lead is adapted to be used to deliver neural stimulation to a neural target. A test lead cable for an external analyzer is connected to at least one terminal of the neural stimulation lead. Test neural stimulation is delivered using an external neural stimulation through the test lead cable and the neural stimulation to the neural target. A physiologic response to the test neural stimulation is monitored.

This Summary is an overview of some of the teachings of the present application and not intended to be an exclusive or exhaustive treatment of the present subject matter. Further details about the present subject matter are found in the detailed description and appended claims. Other aspects will be apparent to persons skilled in the art upon reading and understanding the following detailed description and viewing the drawings that form a part thereof, each of which are not to be taken in a limiting sense. The scope of the present invention is defined by the appended claims and their equivalents.

### BRIEF DESCRIPTION OF THE DRAWINGS

FIG. 1 illustrates an embodiment of a neural stimulation system analyzer.

FIG. 2 illustrates an implantable neural stimulator and an embodiment of a neural stimulation system analyzer.

FIG. 3 illustrates an embodiment of a neural stimulation system analyzer with a test lead cable connected to a bipolar neural stimulation lead and with a sensor cable connected to ECG electrodes to be placed on a patient's skin.

FIG. 4 illustrates a block diagram for an embodiment of a neural stimulation system analyzer.

FIG. 5 illustrates a more detailed block diagram for the neural stimulation system analyzer embodiment illustrated in FIG. 4.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

The present subject matter relates to a neural stimulation system analyzer, which is an external device used acutely during a neural stimulator system implant to test system integrity and titrate therapy. An embodiment of the neural stimulation system analyzer uses two interface cables, where one interface cable is a lead test cable, and the other is a sensor cable. The lead test cable is adapted to be operably attached to an implantable neural stimulation lead for use in testing the lead. In an example where the implantable neural stimulation lead is a bipolar lead, the lead test cable is a bipolar cable. An embodiment of the lead test cable terminates in alligator clips, which can be used to attach the bipolar test cable to the bipolar implantable neural stimulation lead. Other connectors or clamps adapted to quickly make and break an electrical and mechanical connection between the lead test cable and the implantable neural stimulation cable can be used. An embodiment of the sensor cable is a multipolar cable that terminates in two or more ECG button connectors to be placed on the patient's skin.

In some embodiments, the neural stimulation system analyzer measures neural lead impedance to determine system integrity. Some embodiments deliver a burst of neural stimulation and acutely measure a physiologic response, such as heart rate or heart rate variability (HRV) using surface ECG electrodes. Neural stimulation parameters can be adjusted, as necessary, at implant to achieve a desired change in the physiologic parameter and determine the physiologic stimulation threshold. Some device embodiments automatically determine the stimulation threshold by adjusting stimulation parameters and measuring the resulting change in the physiologic parameter.

The neural stimulation system analyzer is adapted to test the integrity of an implantable neural stimulator, and the placement of the stimulator electrodes to capture an autonomic nervous system (ANS) target, such as a vagus nerve. Implantable neural stimulation can deliver vagal modulation to treat a variety of cardiovascular disorders, including heart failure, post-MI remodeling, and hypertension. ANS and some cardiovascular disorders are briefly described below.

Some embodiments of the neural stimulation analyzer deliver test neural stimulation, monitor a physiologic response to the neural stimulation, and titrate parameter(s) of the test neural stimulation as may be necessary to realize the target physiologic response. Amplitude, frequency, pulse duration, duty cycle or other parameters can be adjusted to adjust the neural stimulation intensity. Various device embodiments include a pacing circuit, a sensing circuit, and a controller to communicate with the pacing circuit and the sensing circuit to receive a sensed physiologic response, and automatically adjust an intensity of the test neural stimulation signal until the sensed physiologic response corresponds to a target physiologic response. Thus, for example, a physician can connect the analyzer to the implantable lead, and initiate the start of the analysis, and the analyzer automatically adjusts the stimulation parameter(s) to achieve the desired response. The parameters of the test neural stimulation that realize the target response can be programmed into the implantable neural stimulator.

The ANS regulates “involuntary” organs, while the contraction of voluntary (skeletal) muscles is controlled by somatic motor nerves. Examples of involuntary organs include respiratory and digestive organs, and also include blood vessels and the heart. Often, the ANS functions in an involuntary, reflexive manner to regulate glands, to regulate muscles in the skin, eye, stomach, intestines and bladder, and to regulate cardiac muscle and the muscle around blood vessels, for example.

The ANS includes the sympathetic nervous system and the parasympathetic nervous system. The sympathetic nervous system is affiliated with stress and the “fight or flight response” to emergencies. Among other effects, the “fight or flight response” increases blood pressure and heart rate to increase skeletal muscle blood flow, and decreases digestion to provide the energy for “fighting or fleeing.” The parasympathetic nervous system is affiliated with relaxation and the “rest and digest response” which, among other effects, decreases blood pressure and heart rate, and increases digestion to conserve energy. The ANS maintains normal internal function and works with the somatic nervous system.

The heart rate and force is increased when the sympathetic nervous system is stimulated, and is decreased when the sympathetic nervous system is inhibited (the parasympathetic nervous system is stimulated). An afferent neural pathway conveys impulses toward a nerve center. An efferent neural pathway conveys impulses away from a nerve center.

Stimulating the sympathetic and parasympathetic nervous systems can have effects other than heart rate and blood pressure. For example, stimulating the sympathetic nervous system dilates the pupil, reduces saliva and mucus production, relaxes the bronchial muscle, reduces the successive waves of involuntary contraction (peristalsis) of the stomach and the motility of the stomach, increases the conversion of glycogen to glucose by the liver, decreases urine secretion by the kidneys, and relaxes the wall and closes the sphincter of the bladder. Stimulating the parasympathetic nervous system (inhibiting the sympathetic nervous system) constricts the pupil, increases saliva and mucus production, contracts the bronchial muscle, increases secretions and motility in the stomach and large intestine, increases digestion in the small intention, increases urine secretion, and contracts the wall and relaxes the sphincter of the bladder. The functions associated with the sympathetic and parasympathetic nervous systems are many and can be complexly integrated with each other.

Heart failure refers to a clinical syndrome in which cardiac function causes a below normal cardiac output that can fall below a level adequate to meet the metabolic demand of peripheral tissues. Heart failure may present itself as congestive heart failure (CHF) due to the accompanying venous and pulmonary congestion. Heart failure can be due to a variety of etiologies such as ischemic heart disease.

Hypertension is a cause of heart disease and other related cardiac co-morbidities. Hypertension occurs when blood vessels constrict. As a result, the heart works harder to maintain flow at a higher blood pressure, which can contribute to heart failure. Hypertension generally relates to high blood pressure, such as a transitory or sustained elevation of systemic arterial blood pressure to a level that is likely to induce cardiovascular damage or other adverse consequences. Hypertension has been arbitrarily defined as a systolic blood pressure above 140 mm Hg or a diastolic blood pressure above 90 mm Hg. Consequences of uncontrolled hypertension include, but are not limited to, retinal vascular disease and stroke, left ventricular hypertrophy and failure, myocardial infarction, dissecting aneurysm, and renovascular disease.

Cardiac remodeling refers to a complex remodeling process of the ventricles that involves structural, biochemical, neurohormonal, and electrophysiologic factors, which can result following a myocardial infarction (MI) or other cause of decreased cardiac output. Ventricular remodeling is triggered by a physiological compensatory mechanism that acts to increase cardiac output due to so-called backward failure which increases the diastolic filling pressure of the ventricles and thereby increases the so-called preload (i.e., the degree to which the ventricles are stretched by the volume of blood in the ventricles at the end of diastole). An increase in preload causes an increase in stroke volume during systole, a phenomena known as the Frank-Starling principle. When the ventricles are stretched due to the increased preload over a period of time, however, the ventricles become dilated. The enlargement of the ventricular volume causes increased ventricular wall stress at a given systolic pressure. Along with the increased pressure-volume work done by the ventricle, this acts as a stimulus for hypertrophy of the ventricular myocardium. The disadvantage of dilatation is the extra workload imposed on normal, residual myocardium and the increase in wall tension (Laplace's Law) which represent the stimulus for hypertrophy. If hypertrophy is not adequate to match increased tension, a vicious cycle ensues which causes further and progressive dilatation. As the heart begins to dilate, afferent baroreceptor and cardiopulmonary receptor signals are sent to the vasomotor central nervous system control center, which responds with hormonal secretion and sympathetic discharge. It is the combination of hemodynamic, sympathetic nervous system and hormonal alterations (such as presence or absence of angiotensin converting enzyme (ACE) activity) that ultimately account for the deleterious alterations in cell structure involved in ventricular remodeling. The sustained stresses causing hypertrophy induce apoptosis (i.e., programmed cell death) of cardiac muscle cells and eventual wall thinning which causes further deterioration in cardiac function. Thus, although ventricular dilation and hypertrophy may at first be compensatory and increase cardiac output, the processes ultimately result in both systolic and diastolic dysfunction. It has been shown that the extent of ventricular remodeling is positively correlated with increased mortality in post-MI and heart failure patients.

The present subject matter relates to a neural stimulation analyzer that delivers test neural stimulation to an implantable neural stimulation lead. The neural stimulation system analyzer is adapted to detect a physiologic response to a test neural stimulation to determine whether the test neural stimulation is effective. The monitored physiologic response should be a quick response that indicates that the neural target has been stimulated. For example, heart rate, heart rate variability (HRV) and/or heart rate turbulence (HRT) can be monitored when a test neural stimulation is delivered to the vagus nerve.

HRV relates to the regulation of the sinoatrial node, the natural pacemaker of the heart by the sympathetic and parasympathetic branches of the autonomic nervous system. The time interval between intrinsic ventricular heart contractions changes in response to the body's metabolic need for a change in heart rate and the amount of blood pumped through the circulatory system. For example, during a period of exercise or other activity, a person's intrinsic heart rate will generally increase over a given period of time. However, even on a beat-to-beat basis, that is, from one heart beat to the next, and without exercise, the time interval between intrinsic heart contractions varies in a normal person. These beat-to-beat variations in intrinsic heart rate are the result of proper regulation by the autonomic nervous system on blood pressure and cardiac output; the absence of such variations indicates a possible deficiency in the regulation being provided by the autonomic nervous system. One method for analyzing HRV involves detecting intrinsic ventricular contractions, and recording the time intervals between these contractions, referred to as the R-R intervals, after filtering out any ectopic contractions (ventricular contractions that are not the result of a normal sinus rhythm). This signal of R-R intervals is typically transformed into the frequency-domain, such as by using fast Fourier transform (FFT) techniques, so that its spectral frequency components can be analyzed and divided into low and high frequency bands. For example, the low frequency (LF) band can correspond to a frequency (f) range 0.04 Hz<f<0.15 Hz, and the high frequency (HF) band can correspond to a frequency range 0.15 Hz<f<0.40 Hz. The HF band of the R-R interval signal is influenced only by the parasympathetic/vagal component of the autonomic nervous system. The LF band of the R-R interval signal is influenced by both the sympathetic and parasympathetic components of the autonomic nervous system. Consequently, the ratio LF/HF is regarded as a good indication of the autonomic balance between sympathetic and parasympathetic/vagal components of the autonomic nervous system. An increase in the LF/HF ratio indicates an increased predominance of the sympathetic component, and a decrease in the LF/HF ratio indicates an increased predominance of the parasympathetic component. Thus, in an embodiment in which vagal stimulation is delivered to enhance nerve activity in the vagus nerve, effective vagal stimulation is expected to elicit a parasympathetic response which can be detected by a decrease in the LF/HF ratio. Neural stimulation can also be delivered to inhibit nerve traffic. Neural stimulation to inhibit nerve activity in the vagus nerve is expected to elicit a sympathetic response which can be detected by an increase in the LF/HF ratio. A spectral analysis of the frequency components of the R-R interval signal can be performed using a FFT (or other parametric transformation, such as autoregression) technique from the time domain into the frequency domain. One example of a HRV parameter is SDANN (standard deviation of averaged NN intervals), which represents the standard deviation of the means of all the successive 5 minutes segments contained in a whole recording. Other HRV parameters can be used.

HRT is the physiological response of the sinus node to a premature ventricular contraction (PVC), consisting of a short initial heart rate acceleration followed by a heart rate deceleration. HRT has been shown to be an index of autonomic function, closely correlated to HRV, and is believed to be due to the autonomic baroreflex. The PVC causes a brief disturbance of the arterial blood pressure (low amplitude of the premature beat, high amplitude of the ensuing normal beat), which instantaneously responds in the form of HRT if the autonomic system is healthy, but is either weakened or missing if the autonomic system is impaired. By way of example and not limitation, it has been proposed to quantify HRT using Turbulence Onset (TO) and Turbulence Slope (TS). TO refers to the difference between the heart rate immediately before and after a PVC, and can be expressed as a percentage. For example, if two beats are evaluated before and after the PVC, TO can be expressed as:

$$TO \% = \frac{( {RR}_{+ 1} + {RR}_{+ 2} ) - ( {RR}_{- 2} + {RR}_{- 1} )}{( {RR}_{- 2} + {RR}_{- 1} )} * 100.$$

RR₋₂ and RR₋₁ are the first two normal intervals preceding the PVC and RR₊₁ and RR₊₂ are the first two normal intervals following the PVC. In various embodiments, TO is determined for each individual PVC, and then the average value of all individual measurements is determined. However, TO does not have to be averaged over many measurements, but can be based on one PVC event. Positive TO values indicate deceleration of the sinus rhythm, and negative values indicate acceleration of the sinus rhythm. The number of R-R intervals analyzed before and after the PVC can be adjusted according to a desired application. TS, for example, can be calculated as the steepest slope of linear regression for each sequence of five R-R intervals. In various embodiments, the TS calculations are based on the averaged tachogram and expressed in milliseconds per RR interval. However, TS can be determined without averaging. The number of R-R intervals in a sequence used to determine a linear regression in the TS calculation also can be adjusted according to a desired application. Rules or criteria can be provided for use to select PVCs and for use in selecting valid RR intervals before and after the PVCs. A PVC event can be defined by an R-R interval in some interval range that is shorter than a previous interval by some time or percentage, or it can be defined by an R-R interval without an intervening P-wave (atrial event) if the atrial events are measured. Various embodiments select PVCs only if the contraction occurs at a certain range from the preceding contraction and if the contraction occurs within a certain range from a subsequent contraction. For example, various embodiments limit the HRT calculations to PVCs with a minimum prematurity of 20% and a post-extrasystole interval which is at least 20% longer than the normal interval. Additionally, pre-PVC R-R and post-PVC R-R intervals are considered to be valid if they satisfy the condition that none of the beats are PVCs. One HRT process, for example, excludes RR intervals that are less than a first time duration, that are longer than a second time duration, that differ from a preceding interval by more than a third time duration, or that differ from a reference interval by a predetermined amount time duration or percentage. In an embodiment of such an HRT process with specific values, RR intervals are excluded if they are less than 300 ms, are more than 2000 ms, differ from a preceding interval by more than 200 ms, or differ by more than 20% from the mean of the last five sinus intervals. Various embodiments of the present subject matter provide programmable parameters, such as any of the parameters identified above, for use in selecting PVCs and for use in selecting valid RR intervals before and after the PVCs. Benefits of using HRT to monitor autonomic balance include the ability to measure autonomic balance at a single moment in time. Additionally, unlike the measurement of HRV, HRT assessment can be performed in patients with frequent atrial pacing. Further, HRT analysis provides for a simple, non-processor-intensive measurement of autonomic balance. Thus, data processing, data storage, and data flow are relatively small, resulting in a device with less cost and less power consumption. Also, HRT assessment is faster than HRV, requiring much less R-R data. HRT allows assessment over short recording periods similar in duration to typical neural stimulation burst durations, such as on the order of tens of seconds, for example.

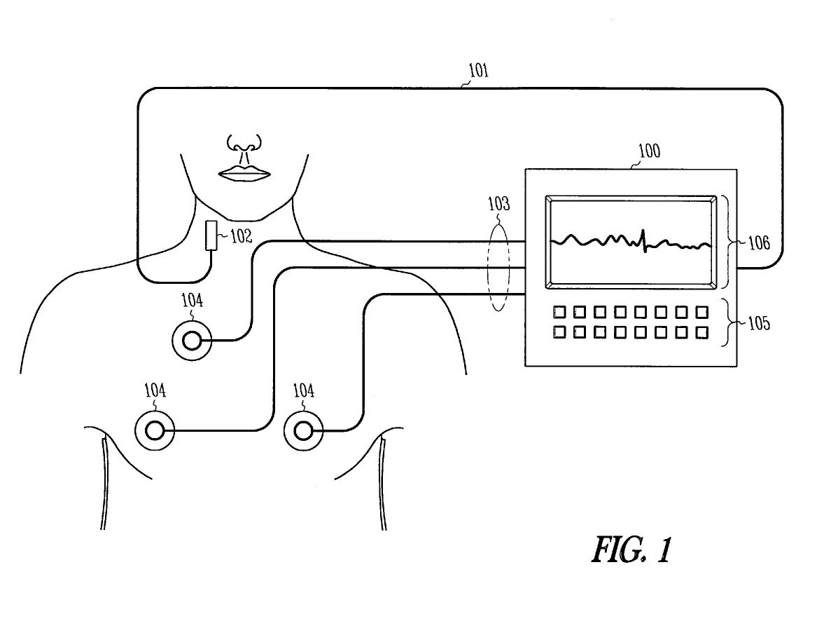

FIG. 1 illustrates an embodiment of a neural stimulation system analyzer. The illustrated neural stimulation system analyzer 100 is an external device. The illustrated external device is adapted to use a test lead cable 101 to temporarily and operationally connect to an implantable neural stimulation lead 102, which will be connected to an implantable pulse generator housing for a neural stimulator (not shown). The illustrated neural stimulation lead is illustrated in the cervical region of the patient, where the right vagus nerve, for example, could be targeted for neural stimulation. Various embodiments intravascularly feed the neural stimulation lead to a position proximate a desired neural target to transvascularly stimulate the neural target. For example, a neural stimulation lead can be fed into an internal jugular vein to stimulate a vagus nerve. Various embodiments transcutaneously tunnel the neural stimulation lead to the desired neural target. The illustrated analyzer is adapted to use at least one sensor cable 103, such as a multipolar sensor cable, connected to two or more ECG electrodes 104 for use in detecting electrical activity of the heart. For example, the ECG electrodes can be used to detect heart rate, HRV, and HRT. Some embodiments use other physiologic parameter sensors such as respiration or blood pressure sensors, either in place of or in addition to, the ECG electrodes.

The neural system analyzer 100 is adapted to deliver neural stimulation to the neural target through the test lead cable 101 and the implantable neural stimulation lead 102. The analyzer 100 is also adapted to sense a physiological response indicative of whether the neural target is being stimulated. The ECG electrodes can detect electrocardiograms, which can be used to detect heart rate. The detected heart rate can be used to perform heart rate variability (HRV) and heart rate turbulence (HRT) tests. Heart rate, HRV and HRT are examples of physiologic measurements that can indicate whether neural stimulation captured a desired target of the autonomic nervous system. Thus, for example, the neural stimulation lead can be appropriately moved to capture the neural target. Some embodiments of the external device verify the integrity of the neural stimulation lead, such as may be performed by testing the impedance of the lead. A high impedance, for example, may indicate a broken conductor in the lead.

The illustrated analyzer 100 includes a user interface with an input 105 such as buttons and an output such as a display 106. The buttons can be used to control the delivery of the test neural stimulation, and the display can be used to show a correlation between the neural stimulation and the monitored physiological response that indicates a successive test stimulation.

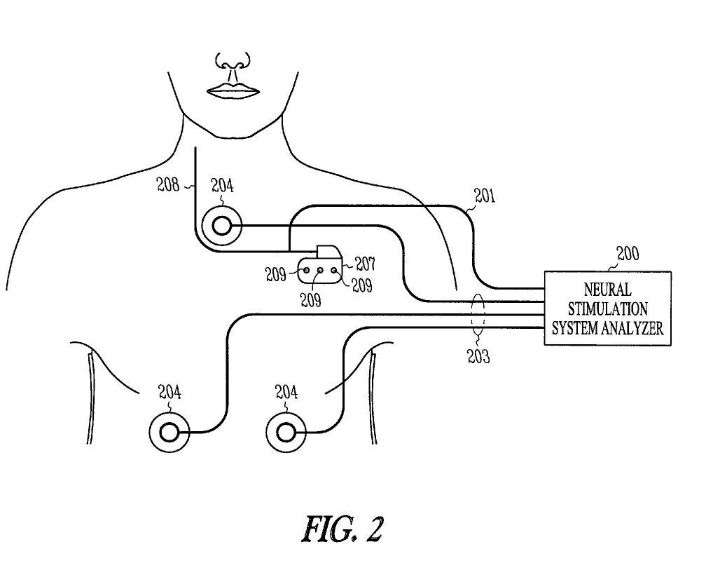

FIG. 2 illustrates an implantable neural stimulator 207 and an embodiment of a neural stimulation system analyzer 200. The illustrated implantable neural stimulator 207 is placed subcutaneously or submuscularly in a patient's chest with lead(s) 208 positioned to stimulate a neural target in the cervical region (e.g. a vagus nerve). The illustrated system provides a lead to the right vagus nerve. The lead could be routed to the left vagus nerve. Some embodiments use leads to stimulate both the left and right vagus nerve. According to various embodiments, neural stimulation lead(s) 208 are subcutaneously tunneled to a neural target, and can have a nerve cuff electrode to stimulate the neural target. Some vagus nerve stimulation lead embodiments are intravascularly fed into a vessel proximate to the neural target, and use electrode(s) within the vessel to transvascularly stimulate the neural target. For example, some embodiments stimulate the vagus using electrode(s) positioned within the internal jugular vein. The neural targets can be stimulated using other energy waveforms, such as ultrasound and light energy waveforms. The illustrated neural stimulation includes leadless ECG electrodes 209 on the housing of the device, which are capable of being used to detect heart rate, for example, to provide feedback for the neural stimulation therapy. At the time of the implantation of the neural stimulator, the test lead cable 201 is temporarily connected to the implanted neural stimulation lead to enable the analyzer to determine an appropriate placement of the lead, and verify the integrity of the stimulation path within the lead. The ECG electrodes, for example, are connected to the analyzer and are also temporarily placed on the patient, enabling the analyzer to verify the capture of the neural target.

Sensor cable(s) 203 connect the analyzer to electrodes 204. These electrodes are used by the analyzer to detect electrical activity of the heart in response to a test neural stimulation delivered through the implantable neural stimulation lead through the test lead 201. For example, the electrodes 204 can be used to detect heart rate, HRV, and/or HRT.

In some embodiments, the implantable neural stimulator is integrated with an implantable cardiac rhythm management device with lead(s) positioned to provide a CRM therapy to a heart. The CRM leads can be used to deliver a cardiac stimulation signal. The CRM leads can be used to pace the heart as part of a bradycardia therapy, an anti-tachycardia or a cardiac resynchronization therapy, for example, to shock the heart as part of an antitachycardia therapy, and to sense cardiac activity. Various embodiments use the CRM lead can also be used to deliver a premature ventricular contraction to perform an HRT analysis.

Some embodiments of the analyzer are adapted to analyze both a neural stimulation system and a cardiac stimulation system. The testing can be done sequentially, as the cardiac and neural leads are implanted. Various cardiac lead embodiments have both pace and sense capabilities, such that the cardiac lead can be used to determine if the test pacing parameters attain a desired response. According to some device embodiments, the pacing circuit is adapted to deliver a test cardiac stimulation signal, and the test lead cable is adapted to electrically connect the pacing circuit and an implantable cardiac stimulation lead. Physiologic feedback is provided using a sensor adapted to sense a physiologic response to cardiac stimulation. The neural stimulation lead and the cardiac stimulation lead can be integrated into one lead.

FIG. 3 illustrates an embodiment of a neural stimulation system analyzer 300 with a test lead cable 301 connected to a bipolar neural stimulation lead 302 and with a sensor cable 303 connected to ECG electrodes 304 to be placed on a patient's skin. The neural stimulation lead 302 has a proximal end 309 for connection to the pulse generator of the implantable neural stimulator, and a distal end 310. The illustrated bipolar neural stimulation lead includes an external covering 311 made from an insulator material, a first electrode 312 illustrated as a tip electrode, and a second electrode 313 illustrated as a ring electrode. The electrodes are not covered by the insulator material. A first wire 314 extends from the first electrode 312 to a first terminal 315 at the proximal end of the lead, and a second wire 316 extends from the second electrode 313 to a second terminal 317 at the proximal end of the lead.

The illustrated test lead cable 301 has two connectors, such as clamps or alligator clips, to connect with the first and second terminals of the neural stimulation lead. The illustrated neural stimulation system analyzer is adapted to generate neural stimulation signals, which are delivered through the test lead cable 301 and through the neural stimulation lead to the first and second electrodes. The illustrated neural stimulation system analyzer is also adapted to test the lead impedance of the neural stimulation lead. A sensor cable 303 includes a sensor for use in detecting a physiologic response to the neural stimulation test to verify capture of the target nerve. The illustrated sensor cable 303 is connected to ECG electrodes 304.

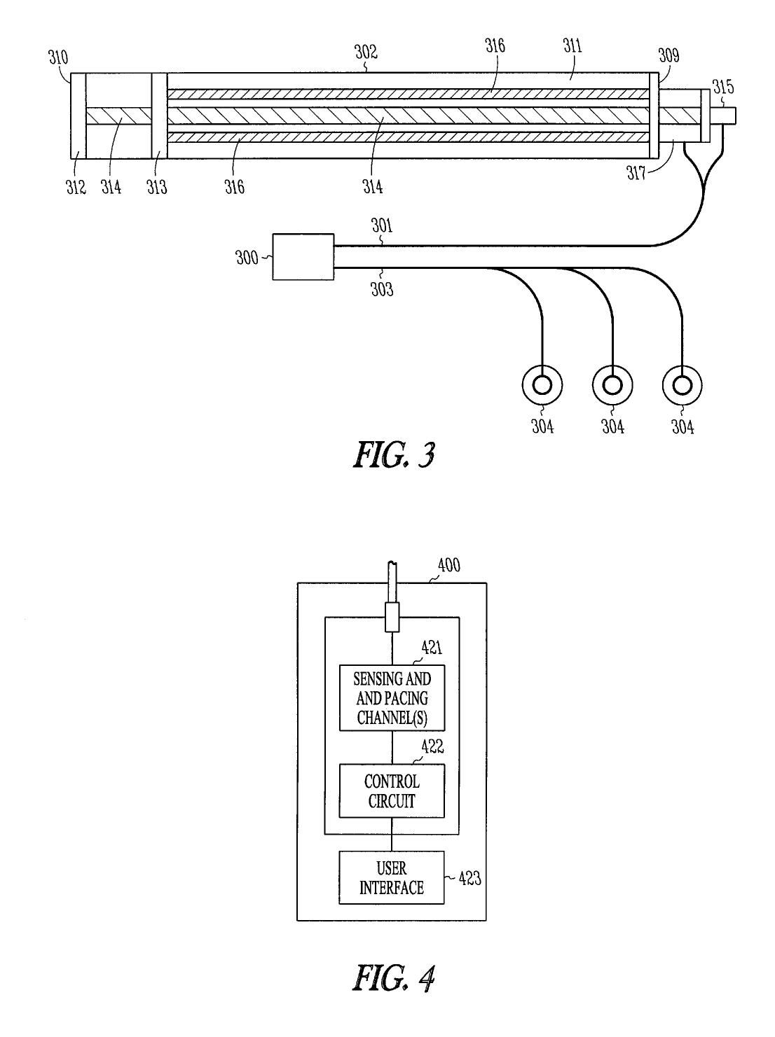

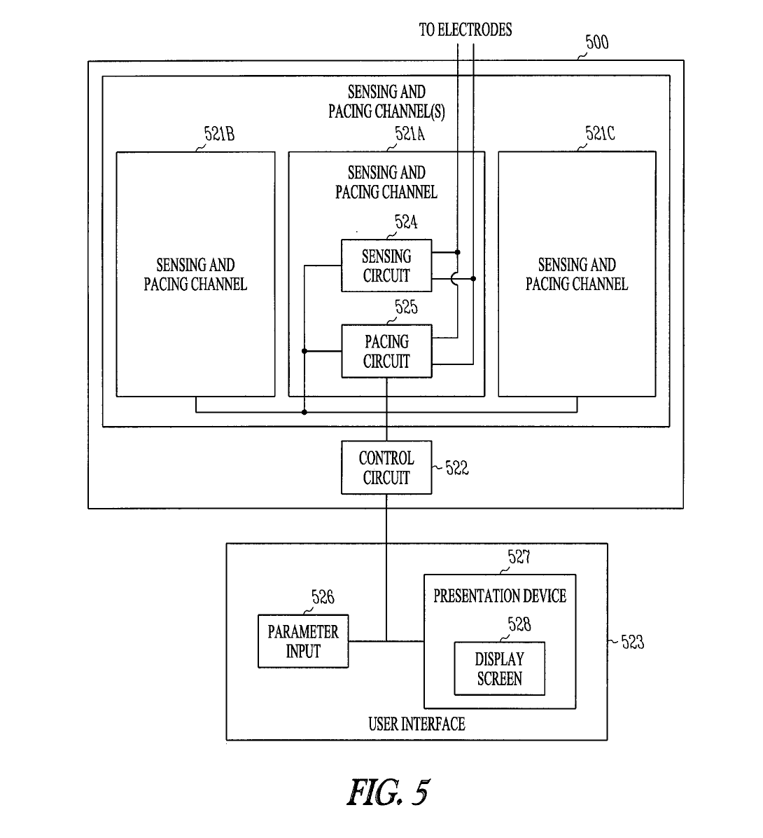

FIG. 4 illustrates a block diagram for an embodiment of a neural stimulation system analyzer 400. The analyzer 400 has an external housing that contains electronic circuitry including sensing and pacing channel(s) 421 and a pacing control circuit 422. The sensing and pacing channel 421 is adapted to generate a neural stimulation burst, and sense a physical parameter responsive to the neural stimulation. The pacing control circuit 422 controls the overall operation of the analyzer 400, including the delivery of the pacing pulses in each sensing and pacing channel. The analyzer 400 also includes a user interface 423, which is electrically connected to the control circuit 422. The user interface 423 allows a user such as a physician or other caregiver to operate the analyzer and observe information acquired by the analyzer. In some embodiments, the user interface is mounted on a housing of the analyzer. An embodiment uses a display screen as a user interface. Other ways to provide feedback to the physician can be used, in addition to or in place of the display screen, such as an audio signal or light. According to some embodiments, the user interface is electrically connected to the electronic circuitry using wires or a cable. The user interface of a computer or a computer-based medical device programmer can be used as user interface. The analyzer can be incorporated into the computer or computer-based medical device programmer. Some analyzer embodiments are configured for detachable attachment to the computer or computer-based medical device programmer.

FIG. 5 illustrates a more detailed block diagram for the neural stimulation system analyzer embodiment illustrated in FIG. 4. The analyzer 500 includes a sensing and pacing channel 521A, pacing control circuit 522, and a user interface 523. The sensing and pacing channel includes a sensing circuit 524 to sense a physiologic response (e.g. heart rate, HRV, or HRT) and a pacing circuit 525 to deliver neural stimulation. The sensing and pacing channel can include multiple channels (e.g. 521B and 521C) to accommodate additional neural stimulation leads or to accommodate more complex electrical arrangements capable of providing various stimulation vectors among the electrodes. The pacing control circuit 522 controls the delivery of pacing pulses to the neural target using a plurality of pacing parameters including user-programmable pacing parameters. These programmable pacing parameters can be evaluated using the analyzer.

The illustrated user interface 523 includes a pacing parameter input 526 and a presentation device 527. The parameter input allows the user to enter and/or adjust the user-programmable pacing parameters. The presentation device includes a display screen 528 for displaying neural stimulation signal and/or physiological sensing signals in real time. Other outputs, such as an audio signal, can be used in addition to or in place of the presentation device to provide an indication of whether the test neural stimulation successfully stimulated a target nerve.

According to various embodiments, the device, as illustrated and described above, is adapted to deliver neural stimulation as electrical stimulation to desired neural targets, such as through one or more stimulation electrodes positioned at predetermined location(s). Other elements for delivering neural stimulation can be used. For example, some embodiments use transducers to deliver neural stimulation using other types of energy, such as ultrasound, light, magnetic or thermal energy.

One of ordinary skill in the art will understand that, the modules and other circuitry shown and described herein can be implemented using software, hardware, and combinations of software and hardware. As such, the terms module and circuitry, for example, are intended to encompass software implementations, hardware implementations, and software and hardware implementations.

The methods illustrated in this disclosure are not intended to be exclusive of other methods within the scope of the present subject matter. Those of ordinary skill in the art will understand, upon reading and comprehending this disclosure, other methods within the scope of the present subject matter. The above-identified embodiments, and portions of the illustrated embodiments, are not necessarily mutually exclusive. These embodiments, or portions thereof, can be combined. In various embodiments, the methods are implemented using a computer data signal embodied in a carrier wave or propagated signal, that represents a sequence of instructions which, when executed by a processor cause the processor to perform the respective method. In various embodiments, the methods are implemented as a set of instructions contained on a computer-accessible medium capable of directing a processor to perform the respective method. In various embodiments, the medium is a magnetic medium, an electronic medium, or an optical medium.

The above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A device to analyze an implantable neural stimulation system that includes an implantable neural stimulation lead for an implantable neural stimulator to be implanted into a patient, the device comprising:
an external housing;
a pacing circuit in the housing, the pacing circuit adapted to deliver a test neural stimulation signal;
at least one test lead cable adapted to electrically connect the pacing circuit and the implantable neural stimulation lead to enable the test neural stimulation signal to be delivered to a neural target through the test lead cable and the implantable neural stimulation lead;
a sensing circuit in the housing;
at least one physiological sensor adapted to sense a physiological response to stimulation of the neural target; and
at least one sensor cable adapted to electrically connect the sensing circuit and the at least one physiological sensor.

2. The device of claim 1, wherein the at least one physiological sensor includes a heart rate sensor to sense a heart rate response to stimulation of a vagus nerve.

3. The device of claim 1, wherein the at least one physiological sensor includes a plurality of ECG electrodes to sense a response to stimulation of a vagus nerve.

4. The device of claim 1, further comprising a controller adapted to communicate with the pacing circuit and the sensing circuit to analyze heart rate variability in response to stimulation of the neural target.

5. The device of claim 1, further comprising a controller adapted to communicate with the pacing circuit and the sensing circuit to analyze heart rate turbulence in response to stimulation of the neural target.

6. The device of claim 1, further comprising a display and a controller adapted to communicate with the pacing circuit, the sensing circuit and the display to provide an indication on the display whether the test neural stimulation signal results in a desired physiological response for stimulation of the neural target.

7. The device of claim 1, wherein the test lead cable includes a bipolar cable adapted to connect to a bipolar neural stimulation lead.

8. The device of claim 1, wherein the test lead cable includes clips adapted to connect to conductor terminals of the neural stimulation lead.

9. The device of claim 1, further comprising a controller to communicate with the pacing circuit and the sensing circuit to receive a sensed physiologic response, and automatically adjust an intensity of the test neural stimulation signal until the sensed physiologic response corresponds to a target physiologic response.

10. The device of claim 1, wherein:
the device is further adapted to analyze a cardiac stimulation system that includes at least one implantable cardiac stimulation lead;
the pacing circuit is adapted to deliver a test cardiac stimulation signal;
the at least one test lead cable is further adapted to electrically connect the pacing circuit and the implantable cardiac stimulation lead; and
the at least one physiological sensor includes a sensor adapted to sense a physiologic response to cardiac stimulation.

11. The device of claim 10, wherein the neural stimulation lead and the cardiac stimulation lead are integrated into one lead.

12. A device to analyze an implantable neural stimulation system that includes an implantable neural stimulation lead for an implantable neural stimulator to be implanted into a patient, comprising:
an external housing;
a pacing circuit in the housing, the pacing circuit adapted to deliver a test neural stimulation signal;
at least one test lead cable adapted to electrically connect the pacing circuit and the implantable neural stimulation lead to enable the test neural stimulation signal to be delivered to a vagus nerve through the test lead cable and the implantable neural stimulation lead, the at least one test lead cable including at least one clip to connect to at least one terminal of the neural stimulation lead;
a sensing circuit in the housing;
a plurality of ECG electrodes adapted for use in detecting an electrocardiogram;
at least one sensor cable adapted to electrically connect the sensing circuit and the plurality of ECG electrodes; and
a controller adapted to communicate with the pacing circuit and the sensing circuit to process the electrocardiogram for use in identifying a response to stimulation of the vagus nerve.

13. The device of claim 12, further comprising a display and a controller adapted to communicate with the pacing circuit, the sensing circuit and the display to provide an indication on the display whether the test neural stimulation signal results in a desired physiological response for stimulation of the vagus nerve.

14. The device of claim 12, wherein the controller is adapted to communicate with the pacing circuit and the sensing circuit to analyze heart rate variability in response to stimulation of the neural target.

15. The device of claim 12, wherein the pacing circuit is further adapted to provide a cardiac stimulation signal.

16. The device of claim 15, wherein the controller is adapted to communicate with the pacing circuit to trigger a premature ventricular pace and communicate with the sensing circuit to analyze heart rate turbulence in response to stimulation of the neural target and the premature ventricular pace.

17. A system for analyzing an implantable neural stimulation system, comprising:
means for connecting a test lead cable for an external analyzer to at least one terminal of an implantable neural stimulation lead;
means for delivering test neural stimulation using an external neural stimulation through the test lead cable and the neural stimulation to a neural target; and
means for monitoring a physiologic response to the test neural stimulation.

18. The system of claim 17, wherein the means for monitoring includes a plurality of ECG electrodes.

19. The system of claim 17, wherein:
the means for monitoring includes means for monitoring a response to vagal stimulation; and
the means for monitoring the response includes means for monitoring heart rate, heart rate variability, or heart rate turbulence.

20. A method for analyzing an implantable neural stimulation system, comprising:
implanting a neural stimulation lead to be used to deliver neural stimulation to a neural target;
connecting a test lead cable for an external analyzer to at least one terminal of the neural stimulation lead;
delivering test neural stimulation using an external neural stimulation through the test lead cable and the neural stimulation to the neural target; and
monitoring a physiologic response to the test neural stimulation.

21. The method of claim 20, wherein implanting the neural stimulation lead includes implanting the neural stimulation lead to be used to deliver neural stimulation to a vagus nerve.

22. The method of claim 20, wherein connecting the test lead cable includes mechanically and electrically attaching the test lead cable to the at least one terminal of the neural stimulation lead.

23. The method of claim 22, wherein connecting the test lead cable includes attaching the test lead cable to the at least one terminal of the neural stimulation lead using at least one clip.

24. The method of claim 20, wherein monitoring the physiologic response to the test neural stimulation includes monitoring heart rate, heart rate variability, or heart rate turbulence.

25. The method of claim 20, further comprising adjusting an implanted position of the neural stimulation lead if the physiologic response to the neural stimulation is not a desired response.

26. The method of claim 20, further comprising:
implanting a cardiac stimulation lead;
connecting a cardiac stimulation test cable for the external analyzer to at least one lead of the cardiac stimulation lead;
delivering test cardiac stimulation; and
monitoring a response to the cardiac stimulation.
